(19) United States
(12) Patent Application Publication
(10) Pub. No.: US 2009/0131657 A1
(43) Pub. Date: May 21, 2009

(54) PROCESS FOR ALKENYLATING CARBOXAMIDES

(75) Inventors: Wolfgang Staffel, Heidelberg (DE); Roland Kessinger, Weinheim (DE); Jochem Henkelmann, Mannheim (DE); Stefan Kashammer, Schifferstadt (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF SE, Ludwigshafen (DE)

(21) Appl. No.: 12/304,669

(22) PCT Filed: Jun. 4, 2007
(86) PCT No.: PCT/EP2007/055444

§ 371 Date: Dec. 12, 2008

(30) Foreign Application Priority Data
Jun. 14, 2006 (DE) 10 2006 028 000.8

(51) Int. Cl. C07D 201/02; C07C 231/12

(52) U.S. Cl. 540/533; 564/205

(57) ABSTRACT

The present invention relates to a process for preparing N-(1-alkenyl)carboxamides of the formula I, which comprises reacting a carboxamide of the formula II with an alkyne of the formula III in the presence of a catalyst selected from among carbonyl complexes, halides and oxides of rhenium, manganese, tungsten, molybdenum, chromium and iron.

The present invention relates to a process for preparing N-(1-alkenyl)carboxamides by reacting a carboxamide with a terminal alkyne.

The addition of carboxamides onto alkynes to produce the corresponding N-alkenyl carboxamides has been known for a long time. Suitable catalysts are strongly basic compounds, in particular potassium salts such as the potassium salt of the carboxamide participating in the reaction (W. Reppe, Liebigs Ann. Chem. 601, 81 (1956)). It is also possible to use alkali metals such as potassium (W. Reppe, Liebigs Ann. Chem. 601, 81 (1956)) or sterically hindered alkali metal alkoxides (WO 89/09210). Furthermore, T. Kondo et al. in J. Chem. Soc. Chem. Commun. 1995, 413, describe the reaction of 1-hexyne with, for example, acetanilide at 180° C. under super-atmospheric pressure over a ruthenium carbonyl catalyst.

In all the processes of the prior art, the yield of corresponding N-alkenyl carboxamides leaves something to be desired and/or the processes are technically very complicated.

It is therefore an object of the present invention to provide a simple process for preparing N-(1-alkenyl)carboxamides which proceeds in high yield.

In addition, the process should be able to be carried out at temperatures at which thermally labile carboxamides and N-(1-alkenyl)carboxamides do not decompose.

The process should allow the reaction of base-labile starting materials or the synthesis of base-labile products.

Finally, the process should be able to be carried out using small amounts of catalyst in order to limit the costs for the catalyst.

It has now surprisingly been found that this object is achieved when a carbonyl complex of rhenium, manganese, tungsten, molybdenum, chromium or iron is used as catalyst.

The present invention accordingly provides a process for preparing N-(1-alkenyl)carboxamides of the formula I

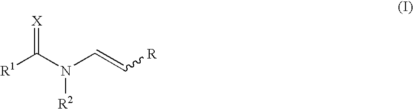

where
R¹ is hydrogen or —C(═X)NR²—CH═CH—R; or
C₁-C₂₀-alkyl, C₂-C₂₀-alkenyl or C₃-C₇-cycloalkyl, where the latter three radicals may optionally be substituted by 1, 2 or 3 radicals selected independently from among phenyl, halogen, hydroxy, C₁-C₄-alkoxy, amino, C₁-C₄-alkylamino, di(C₁-C₄-alkyl)amino, —COR³, —COOR³, —COO—CH═CH—R, —CONR⁴R⁵, —OCOR³, —OCOOR³, —OCONR⁴R⁵, —NR⁴COR⁵, —NR⁴COOR⁵, —NR⁴CONR⁴R⁵ and —C(═X)NR²—CH═CH—R; or
aryl or heteroaryl, where the latter two radicals may optionally be substituted by 1, 2 or 3 radicals selected independently from among C₁-C₄-alkyl, halogen, hydroxy, C₁-C₄-alkoxy, amino, C₁-C₄-alkylamino, di(C₁-C₄-alkyl)amino, —COR³, —COOR³, —COO—CH═CH—R, —CONR⁴R⁵, —OCOR³, —OCOOR³, —OCONR⁴R⁵, —NR⁴COR⁵, —NR⁴COOR⁵, —NR⁴CONR⁴R⁵ and —C(═X)NR²—CH═CH—R;
   R² is hydrogen; or
C₁-C₂₀-alkyl, C₂-C₂₀-alkenyl or C₃-C₇-cycloalkyl, where the latter three radicals may optionally be substituted by 1 or 2 radicals selected independently from among phenyl, halogen and C₁-C₄-alkoxy; or
aryl or heteroaryl, where the latter two radicals may optionally be substituted by 1, 2 or 3 radicals selected independently from among C₁-C₄-alkyl, halogen, C₁-C₄-alkyl, halogen and C₁-C₄-alkoxy; or
C(═X)R⁶;
   R³ is C₁-C₄-alkyl;
   R⁴, R⁵ are each hydrogen or C₁-C₄-alkyl;
   R⁶ is hydrogen; or
C₁-C₂₀-alkyl, C₂-C₂₀-alkenyl or C₃-C₇-cycloalkyl, where the latter three radicals may optionally be substituted by 1, 2 or 3 radicals selected independently from among phenyl, halogen, hydroxy, C₁-C₄-alkoxy, amino, C₁-C₄-alkylamino, di(C₁-C₄-alkyl)amino, —COR³, —COOR³, —COO—CH═CH—R, —CONR⁴R⁵, —OCOR³, —OCOOR³, —OCONR⁴R⁵, —NR⁴COR⁵, —NR⁴COOR⁵, —NR⁴CONR⁴R⁵ and —C(═X)NR²—CH═CH—R; or
aryl or heteroaryl, where the latter two radicals may optionally be substituted by 1, 2 or 3 radicals selected independently from among C₁-C₄-alkyl, halogen, hydroxy, C₁-C₄-alkoxy, amino, C₁-C₄-alkylamino, di(C₁-C₄-alkyl)amino, —COR³, —COOR³, —COO—CH═CH—R, —CONR⁴R⁵, —OCOR³, —OCOOR³, —OCONR⁴R⁵, —NR⁴COR⁵, —NR⁴COOR⁵, —NR⁴CONR⁴R⁵ and —C(═X)NR²—CH═CH—R;
   or
   R¹ and R² together form a —(CH₂)ₙ—, —(CH₂)ₘ—Y—(CH₂)ₒ— or —(CH₂)ₚ—(CH═CH)_{q}— chain,
where
n is 2, 3, 4, 5 or 6;
m is 1, 2 or 3;
o is 1, 2 or 3;
p is 1, 2 or 3;
q is 1 or 2;
Y is O, S or N(C₁-C₄-alkyl);
or
together form a radical (a₁₂), (b₁₂) or (c₁₂)

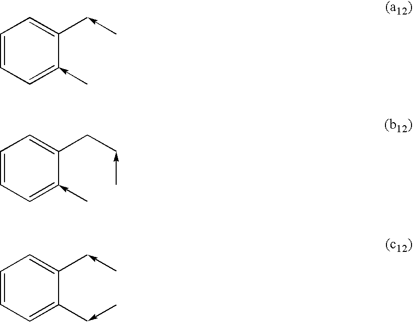

which may optionally be substituted by 1, 2 or 3 radicals selected independently from among C₁-C₄-alkyl, halogen and C₁-C₄-alkoxy;
or
   R¹ and R⁶ together form a —(CH₂)ₙ—, —(CH₂)ₘ—Y—(CH₂)ₒ— or —(CH₂)ₚ—(CH═CH)_{q}— chain,
where
n is 2, 3, 4, 5 or 6;
m is 1, 2 or 3;
o is 1, 2 or 3;
p is 0, 1, 2 or 3;
q is 1 or 2;
Y is O, S or N(C₁-C₄-alkyl);
or
together form arene-1,2-diyl, naphthalene-1,8-diyl or hetarene-1,2-diyl which may optionally be substituted by 1, 2 or 3 radicals selected independently from among C₁-C₄-alkyl, halogen and C₁-C₄-alkoxy;
or
together form a radical (a₁₆), (b₁₆) or (C₁₆)

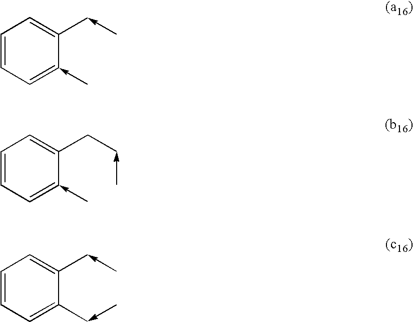

which may optionally be substituted by 1, 2 or 3 radicals selected independently from among C₁-C₄-alkyl, halogen and C₁-C₄-alkoxy;
X is O, S or NR⁷ where R⁷ is hydrogen or C₁-C₈-alkyl;
R is H, C₁-C₈-alkyl, C₃-C₇-cycloalkyl, phenyl-C₁-C₄-alkyl or phenyl, where the phenyl radical of the latter two radicals may optionally be substituted by 1, 2 or 3 radicals selected independently from among C₁-C₄-alkyl, halogen and C₁-C₄ alkoxy;
   by reacting a carboxamide of the formula II

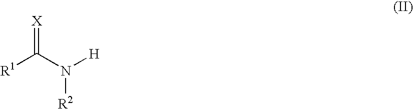

where R¹, R² and X are as defined above and if the radical —(═X)NR²—CH═CH—R is comprised a plurality of times in the N-(1-alkenyl)carboxamide of the formula I, then additionally —C(═X)NHR² in the corresponding position and if the radical —COO—CH═CH—R is comprised one or more times in the N-(1-alkenyl)carboxamide of the formula I, then additionally —COOH in the corresponding position;
with an alkyne of the formula III

H—C≡C—H (III)

where R is as defined above;
in the presence of a catalyst selected from among carbonyl complexes, halides and oxides of ruthenium, manganese, tungsten, molybdenum, chromium or iron.

The alkyl groups can be straight-chain or branched alkyl groups having the indicated number of carbon atoms. Examples of such alkyl groups are methyl, ethyl, n-propyl, i-propyl, n-butyl, i-butyl, sec-butyl, t-butyl, n-hexyl, n-dodecyl, etc., preferably methyl, ethyl, n-propyl, i-propyl, n-butyl and i-butyl.

Examples of C₂-C₂₀-alkenyl groups are ethenyl, 1-propenyl, 2-propenyl, buten-1-yl, buten-2-yl, isobutenyl etc., preferably ethenyl, 2-propenyl or buten-2-yl.

Examples of C₃-C₇-cycloalkyl groups are cyclopropyl, cyclobutyl, cyclopentyl, cyclohexyl and cycloheptyl, preferably cyclopentyl and cyclohexyl.

Halogen is fluorine, chlorine, bromine or iodine, preferably fluorine, chlorine or bromine.

Aryl is, in particular, phenyl or naphthyl, preferably phenyl.

Hetaryl is, in particular, furyl, thienyl, isoxazolyl, isothiazolyl, oxazolyl, thiazolyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, pyridyl, pyrimidinyl, pyrazinyl, pyridazinyl, 1,3,4-triazinyl, 1,2,4-triazinyl or tetrazinyl, preferably pyridyl.

Arene-1,2-diyl is, in particular, benzene-1,2-diyl or naphthalene-2,3-diyl, preferably benzene-1,2-diyl.

Hetarene-1,2-diyl is, in particular, pyridine-2,3-diyl.

The alkyl radicals in the radicals C₁-C₄-alkoxy, C₁-C₄-alkylamino and di(C₁-C₄-alkyl)amino are in each case methyl, ethyl, n-propyl, i-propyl, n-butyl, i-butyl, sec-butyl or t-butyl.

As catalyst, use is made of the carbonyl complexes, halides or oxides of rhenium, manganese, tungsten, molybdenum, chromium or iron. For the purposes of the present invention, carbonyl complexes are compounds which have at least one carbonyl group as ligand; the other coordination sites of the respective metal can be occupied by other ligands. Examples of such ligands are mentioned below. For the purposes of the present invention, halides and oxides also include compounds in which one or more coordination sites and/or valences of the respective metal are occupied by a C₁-C₈-alkyl group, and also oxyhalides. An example is CH₃ReO₃.

The catalysts can be present in all oxidation states; in the case of the carbonyl complexes, they are preferably present in the oxidation state 0 or 1.

Preferred catalysts are the carbonyl complexes, oxides or halides of rhenium, manganese or molybdenum, in particular rhenium. The carbonyl complexes of rhenium and of manganese, in particular of rhenium, have been found to be particularly useful. The carbonyl complexes of the abovementioned metals are particularly effective. One or more of the carbonyl groups can be replaced by suitable ligands such as halogens, in particular chlorine or bromine, phosphine ligands such as triphenylphosphine, trimethylphosphine, triethylphosphine, tri-n-butylphosphine, etc., amine ligands such as NH₃, ethylenediamine, etc., alcohol ligands such as phenol, methanol, ethanol, etc., ether ligands, such as tetrahydrofuran (THF) etc., hydrocarbon ligands such as cyclopentadienyl(Cp), pentamethylcyclopentadienyl(pentamethyl-Cp), cycloocta-1,5-diene or acetonitrile etc., or H₂O. Examples of suitable catalysts are Mn₂(CO)₁₀, W(CO)₆, Mo(CO)₆, Cr(CO)₆, Fe(CO)₅ and Fe₂(CO)₉.

Rhenium catalysts have been found to be particularly useful. Examples are rhenium carbonyl complexes such as Re₂(CO)₁₀, Re(CO)₅Cl, Re(CO)₅Br, ReBr(CO)₃(CH₃CN)₂, ReCp(CO)₃, Re(pentamethyl-Cp)(CO)₃, ReCl(CO)₃(CH₃CN)₂, ReBr(CO)₃(THF)₂ and ReCl(CO)₃(THF)₂, rhenium oxides such as Re₂(pentamethyl-Cp)₂O₄, Re(pentamethyl-Cp)₂OCl₂, Re₂O₇ and ReCH₃O₃, rhenium halides such as ReCl₃ or ReBr₃, or ReCp₂ and Re.

A particularly preferred catalyst is Re₂(CO)₁₀.

The reaction can be carried out in a homogenous or heterogeneous liquid phase. If a homogeneous liquid phase is desired, a catalyst which is soluble in the reaction medium or goes into solution during the reaction is used. Such catalysts are, in particular, the carbonyl complexes of the metals which come into question here and also ReCp₂. Heterogeneous catalysts are generally the halides or “pure” oxides of these metals, e.g. Re₂O₇, and also rhenium metal. The heterogeneous catalysts can be used directly, for example in powder form, or applied to a support. Suitable supports are, for example, carbon, zeolites, aluminum oxides, silicon oxides.

In general, the catalyst is used in an amount of from 0.000005 to 1 mol %, preferably from 0.000005 to 0.5 mol %, more preferably from 0.00001 to 0.1 mol % and in particular from 0.00005 to 0.05 mol %, from 0.0001 to 0.05 mol %, from 0.0005 to 0.01 mol % or from 0.001 to 0.01 mol %, in each case based on the number of equivalents of the compound of the formula II. For the present purposes, the expression “equivalents” is based on —C(═X)NHR² groups and —COOH groups of the formula II which can react with the compound of the formula III.

Suitable starting compounds are carboxamides of the formula II in which the radicals R¹, R² and X, both alone and in combination with one another, have the following meanings:

R¹ is hydrogen or —C(═X)NHR²; or
C₁-C₂₀-alkyl, C₂-C₂₀-alkenyl or C₃-C₇-cycloalkyl, where the latter three radicals may optionally be substituted by 1, 2 or 3 radicals selected independently from among phenyl, halogen, hydroxy, C₁-C₄-alkoxy, amino, C₁-C₄-alkylamino, di(C₁-C₄-alkyl)amino, —COR³, —COOR³, —COOH, —CONR⁴R⁵, —OCOR³, —OCOOR³, —OCONR⁴R⁵, —NR⁴COR⁵, —NR⁴COOR⁵, —NR⁴CONR⁴R⁵ and —C(═X)NHR²; or
aryl or heteroaryl, where the latter two radicals may optionally be substituted by 1, 2 or 3 radicals selected independently from among C₁-C₄-alkyl, halogen, hydroxy, C₁-C₄-alkoxy, amino, C₁-C₄-alkylamino, di(C₁-C₄-alkyl)amino, —COR³, —COOR³, —COOH, —CONR⁴R⁵, —OCOR³, —OCOOR³, —OCONR⁴R⁵, —NR⁴COR⁵, —NR⁴COOR⁵, —NR⁴CONR⁴R⁵ and —C(═X)NHR²;
preferably
hydrogen or —C(═X)NHR², or
C₁-C₂₀-alkyl, C₂-C₂₀-alkenyl or C₃-C₇-cycloalkyl, where the latter three radicals may optionally be substituted by 1 or 2 radicals selected independently from among phenyl, halogen, C₁-C₄-alkoxy and —C(═X)NHR²; or
aryl or heteroaryl, where the latter two radicals may optionally be substituted by 1, 2 or 3 radicals selected independently from among C₁-C₄-alkyl, halogen and C₁-C₄-alkoxy and —C(═X)NHR²;
in particular
hydrogen; or
C₁-C₂₀-alkyl or C₂-C₂₀-alkenyl, where the latter two radicals may optionally be substituted by a radical selected independently from among phenyl and —C(═X)NHR²; or
phenyl which may optionally be substituted by 1, 2 or 3 radicals selected independently from among C₁-C₄-alkyl, halogen, C₁-C₄-alkoxy and —C(═X)NHR²;
   R² is hydrogen; or
C₁-C₂₀-alkyl, C₂-C₂₀-alkenyl or C₃-C₇-cycloalkyl, where the latter three radicals may optionally be substituted by 1 or 2 radicals selected independently from among phenyl, halogen and C₁-C₄-alkoxy; or
aryl or heteroaryl, where the latter two radicals may optionally be substituted by 1, 2 or 3 radicals selected independently from among C₁-C₄-alkyl, halogen, C₁-C₄-alkyl, halogen and C₁-C₄-alkoxy;
preferably
hydrogen; or
C₁-C₈-alkyl which may optionally be substituted by 1 or 2 radicals selected independently from among phenyl, halogen and C₁-C₄-alkoxy; or
aryl which may optionally be substituted by 1, 2 or 3 radicals selected independently from among C₁-C₄-alkyl, halogen, C₁-C₄-alkyl, halogen and C₁-C₄-alkoxy;
in particular
hydrogen; or
C₁-C₈-alkyl; or
phenyl which may optionally be substituted by 1, 2 or 3 radicals selected independently from among C₁-C₄-alkyl, halogen and C₁-C₄-alkoxy;
   R³ is C₁-C₄-alkyl;
   R⁴, R⁵ are each hydrogen or C₁-C₄-alkyl;

### X is O.

Particularly useful starting compounds are aliphatic carboxamides such as aliphatic monocarboxamides per se (R²=hydrogen), aliphatic N—(C₁-C₈-alkyl)mono-carboxamides (R²═C₁-C₈-alkyl) or aliphatic monocarboxanilides (R²=phenyl) or cycloaliphatic monocarboxamides such as cycloaliphatic monocarboxamides per se (R²=hydrogen).

Examples of aliphatic monocarboxamides per se (R²=hydrogen) are formamide, acetamide, propionamide, butyramide, valeramide, hexanoamide, heptanoamide, octanoamide, nonanoamide, decanoamide, 2-methylpropionamide, 2-methyl-butyramide, 3-methylbutyramide, 2-methylpentanoamide, 2-ethylhexanoamide, 2-propylheptanoamide, pivalamide, neononanoamide, neodecanoamide, neotridecanoamide, stearamide, oleamide, lauramide, palmitamide, acrylamide, methacrylamide, crotonamide, cinnamamide or phenylacetamide.

Examples of aliphatic N—(C₁-C₈-alkyl)monocarboxamides (R²═C₁-C₈-alkyl) are N-methylformamide, N-methylacetamide, N-methylpropionamide, N-methylbutyramide, N-methylvaleramide, N-methylhexanoamide, N-methylheptanoamide, N-methyl-octanoamide, N-methylnonanoamide, N-methyldecanoamide, N-methyl-2-methyl-propionamide, N-methyl-2-methylbutyramide, N-methyl-3-methylbutyramide, N-methyl-2-methylpentanoamide, N-methyl-2-ethylhexanoamide, N-methyl-2-propyl-heptanoamide, N-methylpivalamide, N-methylneononanoamide, N-methylneodecano-amide, N-methylneotridecanoamide, N-methylstearamide, N-methyloleamide, N-methyllauramide, N-methylpalmitamide, N-methylacrylamide, N-methyl-methacrylamide, N-methylcrotonamide, N-methylcinnamamide, N-methylphenyl-acetamide, N-ethylformamide, N-ethylacetamide, N-ethylpropionamide, N-ethylbutyramide, N-ethylvaleramide, N-ethylhexanoamide, N-ethylheptanoamide, N-ethyloctanoamide, N-ethylnonanoamide, N-ethyldecanoamide, N-ethyl-2-methyl-propionamide, N-ethyl-2-methylbutyramide, N-ethyl-3-methylbutyramide, N-ethyl-2-methylpentanoamide, N-ethyl-2-ethylhexanoamide, N-ethyl-2-propylheptanoamide, N-ethylpivalamide, N-ethylneononanoamide, N-ethylneodecanoamide, N-ethylneo-tridecanoamide, N-ethylstearamide, N-ethyloleamide, N-ethyllauramide, N-ethyl-palmitamide, N-ethylacrylamide, N-ethylmethyacrylamide, N-ethylcrotonamide, N-ethyl-cinnamamide or N-ethylphenylacetamide.

Examples of aliphatic monocarboxanilides (R²=phenyl) are N-phenylformamide, N-phenylacetamide, N-phenylpropionamide, N-phenylbutyramide, N-phenylvaleramide, N-phenylhexanoamide, N-phenylheptanoamide, N-phenyloctanoamide, N-phenyl-nonanoamide, N-phenyldecanoamide, N-phenyl-2-methylpropionamide, N-phenyl-2-methylbutyramide, N-phenyl-3-methylbutyramide, N-phenyl-2-methylpentanoamide, N-phenyl-2-ethylhexanoamide, N-phenyl-2-propylheptanoamide, N-phenylpivalamide, N-phenylneononanoamide, N-phenylneodecanoamide, N-phenylneotridecanoamide, N-phenylstearamide, N-phenyloleamide, N-phenyllauramide, N-phenylpalmitamide, N-phenylacrylamide, N-phenylmethacrylamide, N-phenylcrotonamide, N-phenyl-cinnamamide, N-phenylphenylacetamide.

Examples of cycloaliphatic monocarboxamides per se (R²=hydrogen) are cyclo-hexanecarboxamide and cycloheptanecarboxamide, preferably cyclohexane-carboxamide.

Further particularly useful starting compounds are aliphatic polycarboxamides, in particular aliphatic dicarboxamides such as aliphatic dicarboxamides per se (R²=hydrogen), aliphatic N—(C₁-C₈-alkyl)dicarboxamides (R²═C₁-C₈-alkyl) or aliphatic dicarboxanilides (R²=phenyl).

Examples of aliphatic dicarboxamides per se (R²=hydrogen) are oxamide (R¹=—CONH₂), malonamide, succinamide, glutaramide, adipamide, sebacamide, maleamide and fumaramide.

Examples of aliphatic N—(C₁-C₈-alkyl)dicarboxamides (R²═C₁-C₈-alkyl) are bis(N-methyl)oxamide(R¹=—CONHCH₃), bis(N-methyl)malonamide, bis(N-methyl)-succinamide, bis(N-methyl)glutaramide, bis(N-methyl)adipamide, bis(N-methyl)sebacamide, bis(N-methyl)maleamide, bis(N-methyl)fumaramide, bis(N-ethyl)-oxamide (R¹=—CONHCH₃), bis(N-ethyl)malonamide, bis(N-ethyl)succinamide, bis(N-ethyl)glutaramide, bis(N-ethyl)adipamide, bis(N-ethyl)sebacamide, bis(N-ethyl)-maleamide and bis(N-ethyl)fumaramide.

Examples of aliphatic dicarboxanilides (R²=phenyl) are bis(N-phenyl)oxamide (R¹=—CONHCH₃), bis(N-phenyl)malonamide, bis(N-phenyl)succinamide, bis(N-phenyl)glutaramide, bis(N-phenyl)adipamide, bis(N-phenyl)sebacamide, bis(N-phenyl)maleamide and bis(N-phenyl)fumaramide.

Further particularly useful starting compounds are aromatic monocarboxamides such as aromatic monocarboxamides per se (R²=hydrogen), aromatic N—(C₁-C₈-alkyl) monocarboxamides (R²═C₁-C₈-alkyl) or aromatic monocarboxanilides (R²=phenyl).

Examples of aromatic monocarboxamides per se (R²=hydrogen) are benzoamide, 2-chlorobenzoamide, 3-chlorobenzoamide, 4-chlorobenzoamide, 2,3-dichlorobenzo-amide, 2,4-dichlorobenzoamide, 2,6-dichlorobenzoamide, 3,4-dichlorobenzoamide, 2,4,6-trichlorobenzoamide, 2-methylbenzoamide, 3-methylbenzoamide, 4-methyl-benzoamide, 2,3-dimethylbenzoamide, 2,4-dimethylbenzoamide, 2,6-dimethyl-benzoamide, 3,4-dimethylbenzoamide, 2,4,6-trimethylbenzoamide, 2-methoxy-benzoamide, 3-methoxybenzoamide, 4-methoxybenzoamide, 2,3-dimethoxy-benzoamide, 2,4-dimethoxybenzoamide, 2,6-dimethoxybenzoamide, 3,4-dimethoxy-benzoamide, 2,4,6-trimethoxybenzoamide and 3,4,5-trimethoxybenzoamide.

Examples of aromatic N—(C₁-C₈-alkyl) monocarboxamides (R²═C₁-C₈-alkyl) are N-methylbenzoamide, N-methyl-2-chlorobenzoamide, N-methyl-3-chlorobenzoamide, N-methyl-4-chlorobenzoamide, N-methyl-2,3-dichlorobenzoamide, N-methyl-2,4-dichlorobenzoamide, N-methyl-2,6-dichlorobenzoamide, N-methyl-3,4-dichloro-benzoamide, N-methyl-2,4,6-trichlorobenzoamide, N-methyl-2-methylbenzoamide, N-methyl-3-methylbenzoamide, N-methyl-4-methylbenzoamide, N-methyl-2,3-dimethyl-benzoamide, N-methyl-2,4-dimethylbenzoamide, N-methyl-2,6-dimethylbenzoamide, N-methyl-3,4-dimethylbenzoamide, N-methyl-2,4,6-trimethylbenzoamide, N-methyl-2-methoxybenzoamide, N-methyl-3-methoxybenzoamide, N-methyl-4-methoxybenzo-amide, N-methyl-2,3-dimethoxybenzoamide, N-methyl-2,4-dimethoxybenzoamide, N-methyl-2,6-dimethoxybenzoamide, N-methyl-3,4-dimethoxybenzoamide, N-methyl-2,4,6-trimethoxybenzoamide, N-methyl-3,4,5-trimethoxybenzoamide, N-ethylbenzo-amide, N-ethyl-2-chlorobenzoamide, N-ethyl-3-chlorobenzoamide, N-ethyl-4-chloro-benzoamide, N-ethyl-2,3-dichlorobenzoamide, N-ethyl-2,4-dichlorobenzoamide, N-ethyl-2,6-dichlorobenzoamide, N-ethyl-3,4-dichlorobenzoamide, N-ethyl-2,4,6-trichlorobenzoamide, N-ethyl-2-methylbenzoamide, N-ethyl-3-methylbenzoamide, N-ethyl-4-methylbenzoamide, N-ethyl-2,3-dimethylbenzoamide, N-ethyl-2,4-dimethyl-benzoamide, N-ethyl-2,6-dimethylbenzoamide, N-ethyl-3,4-dimethylbenzoamide, N-ethyl-2,4,6-trimethylbenzoamide, N-ethyl-2-methoxybenzoamide, N-ethyl-3-methoxy-benzoamide, N-ethyl-4-methoxybenzoamide, N-methyl-2,3-dimethoxybenzoamide, N-methyl-2,4-dimethoxybenzoamide, N-ethyl-2,6-dimethoxybenzoamide, N-ethyl-3,4-dimethoxybenzoamide, N-ethyl-2,4,6-trimethoxybenzoamide and N-ethyl-3,4,5-tri-methoxybenzoamide.

Examples of aromatic monocarboxanilides (R²=phenyl) are N-phenylbenzoamide, N-phenyl-2-chlorobenzoamide, N-phenyl-3-chlorobenzoamide, N-phenyl-4-chloro-benzoamide, N-phenyl-2,3-dichlorobenzoamide, N-phenyl-2,4-dichlorobenzoamide, N-phenyl-2,6-dichlorobenzoamide, N-phenyl-3,4-dichlorobenzoamide, N-phenyl-2,4,6-trichlorobenzoamide, N-phenyl-2-methylbenzoamide, N-phenyl-3-methyl-benzoamide, N-phenyl-4-methylbenzoamide, N-phenyl-2,3-dimethylbenzoamide, N-phenyl-2,4-dimethylbenzoamide, N-phenyl-2,6-dimethylbenzoamide, N-phenyl-3,4-dimethylbenzoamide, N-phenyl-2,4,6-trimethylbenzoamide, N-phenyl-2-methoxy-benzoamide, N-methyl-3-methoxybenzoamide, N-phenyl-4-methoxybenzoamide, N-methyl-2,3-dimethoxybenzoamide, N-phenyl-2,4-dimethoxybenzoamide, N-methyl-2,6-dimethoxybenzoamide, N-phenyl-3,4-dimethoxybenzoamide, N-methyl-2,4,6-tri-methoxybenzoamide.

Further particularly useful starting compounds are aromatic polycarboxamides such as aromatic dicarboxamides, tricarboxamides, tetracarboxamides, pentacarboxamides or hexacarboxamides per se (R²=hydrogen), preferably aromatic dicarboxamides per se (R²=hydrogen), aromatic N—(C₁-C₈-alkyl)dicarboxamides (R²═C₁-C₈-alkyl), aromatic dicarboxanilides (R²=phenyl).

Examples of aromatic dicarboxamides per se (R²=hydrogen) are phthalamide, isophthalamide and terephthalamide.

Examples of aromatic N—(C₁-C₈-alkyl)dicarboxamides (R²═C₁-C₈-alkyl) are bis(N-methyl)phthalamide, bis(N-methyl)isophthalamide, bis(N-methyl)terephthalamide, bis(N-ethyl)phthalamide, bis(N-ethyl)isophthalamide and bis(N-ethyl)terephthalamide.

Examples of aromatic dicarboxanilides (R²=phenyl) are bis(N-phenyl)phthalamide, bis(N-phenyl)isophthalamide, bis(N-phenyl)terephthalamide.

Further suitable starting compounds are carboxamides of the formula II in which the radicals R¹, R² and X, both alone and in combination, have the following meanings:

R¹ is hydrogen or —C(═X)NHR²; or
C₁-C₂₀-alkyl, C₂-C₂₀-alkenyl or C₃-C₇-cycloalkyl, where the latter three radicals may optionally be substituted by 1, 2 or 3 radicals selected independently from among phenyl, halogen, hydroxy, C₁-C₄-alkoxy, amino, C₁-C₄-alkylamino, di(C₁-C₄-alkyl)amino, —COR³, —COOR³, —COOH, —CONR⁴R⁵, —OCOR³, —OCOOR³, —OCONR⁴R⁵, —NR⁴COR⁵, —NR⁴COOR⁵, —NR⁴CONR⁴R⁵ and —C(═X)NHR²; or
aryl or heteroaryl, where the latter two radicals may optionally be substituted by 1, 2 or 3 radicals selected independently from among C₁-C₄-alkyl, halogen, hydroxy, C₁-C₄-alkoxy, amino, C₁-C₄-alkylamino, di(C₁-C₄-alkyl)amino, —COR³, —COOR³, —COOH, —CONR⁴R⁵, —OCOR³, —OCOOR³, —OCONR⁴R⁵, —NR⁴COR⁵, —NR⁴COOR⁵, —NR⁴CONR⁴R⁵ and —C(═X)NHR²;
preferably
hydrogen, or
C₁-C₂₀-alkyl or C₂-C₂₀-alkenyl, where the latter two radicals may optionally be substituted by 1 or 2 radicals selected independently from among phenyl, halogen and C₁-C₄-alkoxy; or
aryl or heteroaryl, where the latter two radicals may optionally be substituted by 1, 2 or 3 radicals selected independently from among C₁-C₄-alkyl, halogen and C₁-C₄-alkoxy;
in particular
hydrogen; or
C₁-C₂₀-alkyl or C₂-C₂₀-alkenyl, where the latter two radicals may optionally be substituted by phenyl; or
phenyl which may optionally be substituted by 1, 2 or 3 radicals selected independently from among C₁-C₄-alkyl, halogen and C₁-C₄-alkoxy;

### R² is C(═X)R⁶;

R³ is C₁-C₄-alkyl;
R⁴, R⁵ is hydrogen or C₁-C₄-alkyl;
R⁶ is hydrogen; or
C₁-C₂₀-alkyl, C₂-C₂₀-alkenyl or C₃-C₇-cycloalkyl, where the latter three radicals may optionally be substituted by 1, 2 or 3 radicals selected independently from among phenyl, halogen, hydroxy, C₁-C₄-alkoxy, amino, C₁-C₄-alkylamino, di(C₁-C₄-alkyl)amino, —COR³, —COOR³, —COOH, —CONR⁴R⁵, —OCOR³, —OCOOR³, —OCONR⁴R⁵, —NR⁴COR⁵, —NR⁴COOR⁵, —NR⁴CONR⁴R⁵ and —C(═X)NHR²; or
aryl or heteroaryl, where the latter two radicals may optionally be substituted by 1, 2 or 3 radicals selected independently from among C₁-C₄-alkyl, halogen, hydroxy, C₁-C₄-alkoxy, amino, C₁-C₄-alkylamino, di(C₁-C₄-alkyl)amino, —COR³, —COOR³, —COOH, —CONR⁴R⁵, —OCOR³, —OCOOR³, —OCONR⁴R⁵, —NR⁴COR⁵, —NR⁴COOR⁵, —NR⁴CONR⁴R⁵ and —C(═X)NHR²;
preferably
hydrogen; or
C₁-C₂₀-alkyl or C₂-C₂₀-alkenyl, where the latter two radicals may optionally be substituted by 1 or 2 radicals selected independently from among phenyl, halogen and C₁-C₄-alkoxy; or
aryl or heteroaryl, where the latter two radicals may optionally be substituted by 1, 2 or 3 radicals selected independently from among C₁-C₄-alkyl, halogen and C₁-C₄-alkoxy;
in particular
C₁-C₂₀-alkyl or C₂-C₂₀-alkenyl, where the latter two radicals may optionally be substituted by phenyl; or
phenyl which may optionally be substituted by 1, 2 or 3 radicals selected independently from among C₁-C₄-alkyl, halogen and C₁-C₄-alkoxy;

### X is O.

Particularly useful compounds are aliphatic carboximides such as N,N-bisformylamine, N,N-bisacetylamine or N,N-bispropionylamine.

Particularly useful compounds are aromatic carboximides such as N,N-bisbenzoyl-amine.

Particularly useful compounds are mixed aliphatic-aromatic carboximides such as N-benzoyl-N-formylamine, N-acetyl-N-benzoylamine or N-benzoyl-N-propionylamine.

Further suitable starting compounds are carboxamides of the formula II in which the radicals R¹, R² and X, either alone or in combination with one another, have the following meanings:

R¹ and R² together form a —(CH₂)ₙ—, —(CH₂)ₘ—Y—(CH₂)ₒ— or —(CH₂)ₚ—(CH═CH)_{q}— chain,
where
n is 2, 3, 4, 5 or 6;
m is 1, 2 or 3;
o is 1, 2 or 3;
p is 1, 2 or 3;
q is 1 or 2;
Y is O, S or N(C₁-C₄-alkyl);
preferably a —(CH₂)ₙ— chain where n=2, 3, 4 or 5 or a —(CH₂)ₚ—(CH═CH)_{q}— chain where p=0 and q=2;
or
together form a radical (a₁₂), (b₁₂) or (c₁₂)

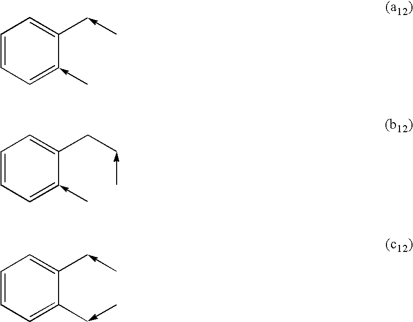

which may optionally be substituted by 1, 2 or 3 radicals selected independently from among C₁-C₄-alkyl, halogen and C₁-C₄-alkoxy;
the compounds of the formula II are preferably compounds of the formula IIa₁₂₋₁, IIa₁₂₋₂, IIb₁₂₋₁, IIb₁₂₋₂ or IIc₁₂₋₁

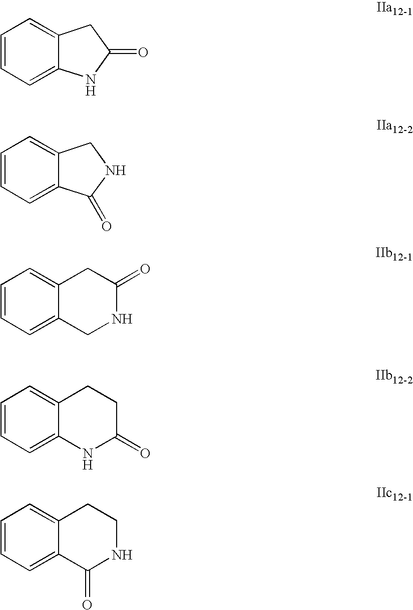

### X is O.

Particularly useful compounds are cyclic carboxamides such as 2-pyrrolidone, pentane-5-lactam, 2-(1H)-pyridone, caprolactam (=azepan-2-one), in particular 2-pyrrolidone and caprolactam, preferably 2-pyrrolidone.

Further suitable starting compounds are carboxamides of the formula II in which the radicals R¹, R⁶ and X, either alone or in combination with one another, have the following meanings:

R¹ and R⁶ together form a —(CH₂)ₙ—, —(CH₂)ₘ—Y—(CH₂)ₒ— or —(CH₂)ₚ—(CH═CH)_{q}— chain,
where
n is 2, 3, 4, 5 or 6;
m is 1, 2 or 3;
0 is 1, 2 or 3;
p is 0, 1, 2 or 3;
q is 1 or 2;
Y is O, S or N(C₁-C₄-alkyl);
preferably a —(CH₂)ₙ— chain where n=2, 3, 4 or 5 or a —(CH₂)ₚ—(CH═CH)_{q}— chain where p=0 and q=1;
or
together form arene-1,2-diyl, naphthalene-1,8-diyl or hetarene-1,2-diyl, which may optionally be substituted by 1, 2 or 3 radicals selected independently from among C₁-C₄-alkyl, halogen and C₁-C₄-alkoxy;
preferably benzene-1,2-diyl or naphthalene-1,8-diyl;
or
together form a radical (a₁₆), (b₁₆) or (c₁₆)

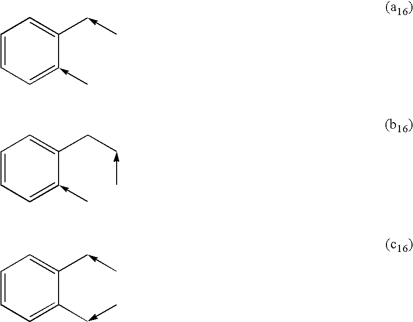

which may optionally be substituted by 1, 2 or 3 radicals selected independently from among C₁-C₄-alkyl, halogen and C₁-C₄-alkoxy;
preferably the compound IIa₁₆₋₁;

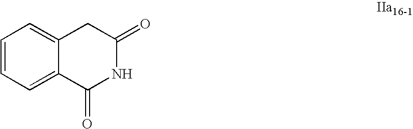

### X is O.

Particularly useful compounds are cyclic biscarboxamides such as fumarimide, succinimide, maleimide, phthalimide, in particular phthalimide.

Suitable starting compounds of the formula III are, for example, acetylene, propyne, 1-butyne, 1-pentyne, 1-hexyne and phenylacetylene, with particular preference being given to using acetylene.

The ratio of compound of the formula II to compound of the formula III can be chosen within a wide range. In general, however, an excess of compound of the formula III is used, in particular an excess of from 0.1 to 20 mol %, based on the compound of the formula II. If the compound of the formula I comprises two or more groups —C(═X)NR²—CH═CH—R and/or one or more groups —COOCH═CHR, the excess is calculated per C(═X)NR²—CH═CH—R or —COOCH═CHR group, i.e. per equivalent of the compound of the formula II.

The reaction is generally carried out in a suitable inert solvent. If the compound of the formula II is liquid at the temperature employed, a solvent can also be dispensed with. Suitable inert solvents are aliphatic and aromatic hydrocarbons such as pentane, hexane, heptane, toluene, xylene, etc., ethers such as tetrahydrofuran or dioxane, chlorinated hydrocarbons such as methylene chloride, 1,2-dichloroethane or chlorobenzene, acetonitrile, dimethylformamide, dimethyl sulfoxide, N-methyl-pyrrolidone or polyethylene glycols or mixtures thereof.

The reaction temperature can be chosen freely within a wide range. It is generally selected so that rapid reaction occurs without starting compounds or the product decomposing. In general, the reactions are carried out at a temperature of less than 250° C. The temperature is usually in the range from 70 to 230° C., in particular from 110 to 210° C., preferably from 130 to 190° C., from 150 to 180° C., particularly preferably from 160 to 170° C.

Depending on the alkyne of the formula III which is used and on any solvent used, the reaction can be carried out under superatmospheric pressure or under atmospheric pressure. If superatmospheric pressure is employed, the reaction is usually carried out at a pressure of from 1 to 50 bar (absolute), with preference being given to setting a pressure of from 1 to 30 bar (absolute), preferably from 2 to 20 bar and in particular from 5 to 25 bar or from 10 to 20 bar. The reaction with acetylene is preferably carried out under superatmospheric pressure. The pressure can, for example, be set by means of the compound of the formula III employed and/or an inert gas such as nitrogen. If the reaction is carried out in the presence of an inert gas, the pressure can also be increased, in particular up to 100 bar, preferably up to 50 bar. The reaction time is usually in the range from 0.01 to 72 hours, in particular from 0.1 to 48 hours.

It is also possible to add, if appropriate, reaction-promoting additives such as zinc acetate, lithium salts, for example LiCl, Lewis acids such as BF₃, etc., Lewis bases such as triethylamine, pyridine, 1,5-diazabicyclo[4.3.0]non-5-ene etc., substances which react with the catalyst at the CO and can thereby create free coordination sites, e.g. trimethylamine N-oxide.

The reaction can be carried out batchwise, continuously or by the semibatch method. The work-up is carried out in a customary manner, advantageously by distilling off the desired carboxamide of the formula I. The catalyst remains in the bottoms and can, if appropriate, be reused. The reaction and/or the work-up, in particular the purifying distillation, can advantageously be carried out in the presence of a polymerization inhibitor. As polymerization inhibitors, it is possible to use, for example, hydroquinone, hydroquinone monomethyl ether, 2,5-di-t-butylhydroquinone, 2,6-di-t-butyl-p-cresol, nitroso compounds such as isoacryl nitrate, nitrosodiphenylamino of N-nitroso-cyclohexylhydroxylamine, methylene blue, phenothiazine, tannic acid or diphenylamine. The polymerization inhibitors are used in amounts of from 1 to 10 000 ppm, in particular from 100 to 1000 ppm, in each case based on the total batch.

A particular embodiment comprises the reaction of compounds of the formula II in which R¹ and R² form a —(CH₂)₃— chain and X is oxygen with acetylene. This reaction is preferably carried out at a temperature in the range from 70 to 220° C., in particular from 120 to 190° C. or from 150 to 170° C. The catalyst is used, in particular, in an amount of from 0.00001 to 0.1 mol %, in particular 0.0001 to 0.01 mol %, based on the carboxamide of the formula II.

A further particular embodiment comprises the reaction of phthalimide (compounds of the formula II in which R¹ and R⁶ together form benzene-1,2-diyl and X is oxygen) with acetylene. This reaction is preferably carried out at a temperature in the range from 70 to 220° C., in particular from 120 to 190° C. or from 150 to 170° C. The catalyst is used, in particular, in an amount of from 0.00001 to 0.1 mol %, in particular from 0.0001 to 0.01 mol %, based on the phthalimide.

The following examples illustrate the invention without restricting its scope. The GC analyses (GC: gas chromatography) were carried out on a capillary column provided with a Carbowax (polyethylene glycol) film, e.g. DB Wax from J & W Scientific.

### EXAMPLES

### Example 1

A mixture of 2.65 g (59 mmol) of formamide, 0.5 g (0.77 mmol) of Re₂(CO)₁₀ and 20.7 g of dioxane were subjected to vinylation at 170° C. under a nitrogen pressure of 2 bar and an acetylene pressure of 18 bar for 0.2 h. Vinylformamide could be detected by means of GC analysis.

### Example 2

### Without Polymerization Inhibitor

A mixture of 10.0 g (118 mmol) of 2-pyrrolidone, 0.5 g (0.77 mmol) of Re₂(CO)₁₀ and 17.4 g of toluene were subjected to vinylation at 170° C. under a nitrogen pressure of 2 bar and an acetylene pressure of 18 bar for 0.2 h. The yield of N-vinyl-2-pyrrolidone determined by GC analysis was 95%.

### Example 3

### With Polymerization Inhibitor

A mixture of 10.0 g (118 mmol) of 2-pyrrolidone, 0.5 g (0.77 mmol) of Re₂(CO)₁₀, 53 mg (0.24 mmol) of di-tert-butyl-p-cresol and 17.4 g of toluene were subjected to vinylation at 170° C. under a nitrogen pressure of 2 bar and an acetylene pressure of 18 bar for 0.2 h. The yield of N-vinyl-2-pyrrolidone determined by GC analysis was 98%.

### Example 4

A mixture of 6.6 g (58 mmol) of caprolactam, 0.5 g (0.77 mmol) of Re₂(CO)₁₀ and 17.4 g of toluene were subjected to vinylation at 170° C. under a nitrogen pressure of 2 bar and an acetylene pressure of 18 bar for 0.2 h. N-Vinylcaprolactam could be detected by means of GC analysis.

### Example 5

A mixture of 29.4 g (200 mmol) of phthalimide, 652 mg (1.00 mmol) of Re₂(CO)₁₀ and 59.0 g of dioxane were subjected to vinylation at 160° C. under a nitrogen pressure of 2 bar and an acetylene pressure of 18 bar for 0.2 h. The yield of N-vinylphthalimide determined by GC analysis was 94%.

1. A process for preparing N-(1-alkenyl)carboxamides of the formula I 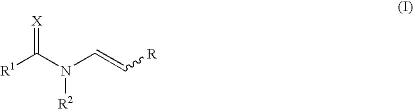
where
R¹ is hydrogen or —C(═X)NR²—CH═CH—R; or
C₁-C₂₀-alkyl, C₂-C₂₀-alkenyl or C₃-C₇-cycloalkyl, where the latter three radicals may optionally be substituted by 1, 2 or 3 radicals selected independently from among phenyl, halogen, hydroxy, C₁-C₄-alkoxy, amino, C₁-C₄-alkylamino, di(C₁-C₄-alkyl)amino, —COR³, —COOR³, —COO—CH═CH—R, —CONR⁴R⁵, —OCOR³, —OCOOR³, —OCONR⁴R⁵, —NR⁴COR⁵, —NR⁴COOR⁵, —NR⁴CONR⁴R⁵ and —C(═X)NR²—CH═CH—R; or
aryl or heteroaryl, where the latter two radicals may optionally be substituted by 1, 2 or 3 radicals selected independently from among C₁-C₄-alkyl, halogen, hydroxy, C₁-C₄-alkoxy, amino, C₁-C₄-alkylamino, di(C₁-C₄-alkyl)amino, —COR³, —COOR³, —COO—CH═CH—R, —CONR⁴R⁵, —OCOR³, —OCOOR³, —OCONR⁴R⁵, —NR⁴COR⁵, —NR⁴COOR⁵, —NR⁴CONR⁴R⁵ and —C(═X)NR²—CH═CH—R;
R² is hydrogen; or
C₁-C₂₀-alkyl, C₂-C₂₀-alkenyl or C₃-C₇-cycloalkyl, where the latter three radicals may optionally be substituted by 1 or 2 radicals selected independently from among phenyl, halogen and C₁-C₄-alkoxy; or
aryl or heteroaryl, where the latter two radicals may optionally be substituted by 1, 2 or 3 radicals selected independently from among C₁-C₄-alkyl, halogen, C₁-C₄-alkyl, halogen and C₁-C₄-alkoxy; or
C(═X)R⁶;
R³ is C₁-C₄-alkyl;
R⁴, R⁵ is hydrogen or C₁-C₄-alkyl;
R⁶ is hydrogen; or
C₁-C₂₀-alkyl, C₂-C₂₀-alkenyl or C₃-C₇-cycloalkyl, where the latter three radicals may optionally be substituted by 1, 2 or 3 radicals selected independently from among phenyl, halogen, hydroxy, C₁-C₄-alkoxy, amino, C₁-C₄-alkylamino, di(C₁-C₄-alkyl)amino, —COR³, —COOR³, —COO—CH═CH—R, —CONR⁴R⁵, —OCOR³, —OCOOR³, —OCONR⁴R⁵, —NR⁴COR⁵, —NR⁴COOR⁵, NR⁴CONR⁴R⁵ and —C(═X)NR²—CH═CH—R; or
aryl or heteroaryl, where the latter two radicals may optionally be substituted by 1, 2 or 3 radicals selected independently from among C₁-C₄-alkyl, halogen, hydroxy, C₁-C₄-alkoxy, amino, C₁-C₄-alkylamino, di(C₁-C₄-alkyl)amino, —COR³, —COOR³, —COO—CH═CH—R, —CONR⁴R⁵, —OCOR³, —OCOOR³, —OCONR⁴R⁵, —NR⁴COR⁵, —NR COOR⁵, —NR⁴CONR⁴R⁵ and —C(═X)NR²—CH═CH—R;
or
R¹ and R² together form a —(CH₂)ₙ—, —(CH₂)ₘ—Y—(CH₂)ₒ— or —(CH₂)ₚ—(CH═CH)_{q}, chain,
where
n is 2, 3, 4, 5 or 6;
m is 1, 2 or 3;
o is 1, 2 or 3;
p is 1, 2 or 3;
q is 1 or 2;
Y is O, S or N(C₁-C₄-alkyl);
or
together form a radical (a₁₂), (b₁₂) or (c₁₂) 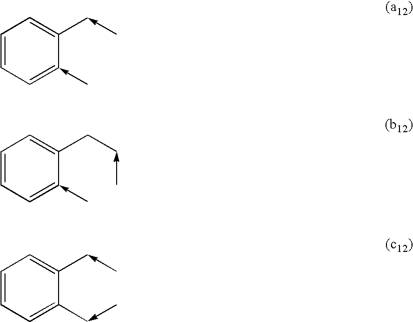
which may optionally be substituted by 1, 2 or 3 radicals selected independently from among C₁-C₄-alkyl, halogen and C₁-C₄-alkoxy;
or
R¹ and R⁶ together form a —(CH₂)ₙ—, —(CH₂)ₘ—Y—(CH₂)ₒ— or —(CH₂)ₚ—(CH═CH)_{q}— chain,
where
n is 2, 3, 4, 5 or 6;
m is 1, 2 or 3;
o is 1, 2 or 3;
p is 0, 1, 2 or 3;
q is 1 or 2;
Y is O, S or N(C₁-C₄-alkyl);
or
together form arene-1,2-diyl, naphthalene-1,8-diyl or hetarene-1,2-diyl which may optionally be substituted by 1, 2 or 3 radicals selected independently from among C₁-C₄-alkyl, halogen and C₁-C₄-alkoxy;
or
together form a radical (a₁₆), (b₁₆) or (c₁₆) 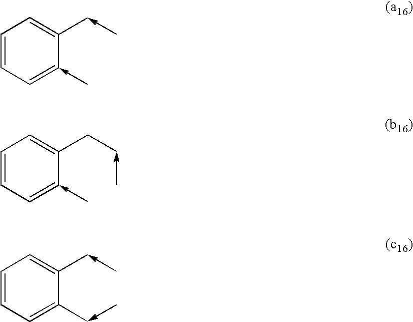
which may optionally be substituted by 1, 2 or 3 radicals selected independently from among C₁-C₄-alkyl, halogen and C₁-C₄-alkoxy;
X is O, S or NR⁷ where R⁷ is hydrogen or C₁-C₈-alkyl;
R is H, C₁-C₈-alkyl, C₃-C₇-cycloalkyl, phenyl-C₁-C₄-alkyl or phenyl, where the phenyl radical of the latter two radicals may optionally be substituted by 1, 2 or 3 radicals selected independently from among C₁-C₄-alkyl, halogen and C₁-C₄ alkoxy;
which comprises reacting a carboxamide of the formula II 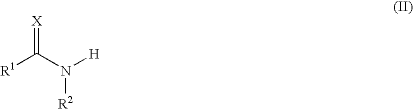
where R¹, R² and X are as defined above and if the radical —(═X)NR²—CH═CH—R is comprised a plurality of times in the N-(1-alkenyl)carboxamide of the formula I, then additionally —C(═X)NHR² in the corresponding position and if the radical —COO—CH═CH—R is comprised one or more times in the N-(1-alkenyl)carboxamide of the formula I, then additionally —COOH in the corresponding position;
with an alkyne of the formula III
H—C≡C—H (III)
where R is as defined above;
in the presence of a catalyst selected from among carbonyl complexes, halides and oxides of ruthenium, manganese, tungsten, molybdenum, chromium or iron and rhenium Cp₂ and rhenium metal.

2. The process according to claim 1, wherein the catalyst is selected from among carbonyl complexes of ruthenium and manganese.

3. The process according to claim 2, wherein Re₂(CO)₁₀ is used as catalyst.

4. The process according to claim 1, wherein the catalyst is used in an amount of from 0.000005 to 1 mol %, based on equivalents of the compound of the formula II.

5. The process according to claim 1, wherein the compound of the formula III is selected from among acetylene, propyne, 1-butyne, 1-pentyne, 1-hexyne, and phenylacetylene.

6. The process according to claim 5, wherein acetylene is used as compound of the formula III.

7. The process according to claim 1, wherein a compound of the formula II in which
R¹ is hydrogen or —C(═X)NHR²; or
C₁-C₂₀-alkyl, C₂-C₂₀-alkenyl or C₃-C₇-cycloalkyl, where the latter three radicals may optionally be substituted by 1, 2 or 3 radicals selected independently from among phenyl, halogen, hydroxy, C₁-C₄-alkoxy, amino, C₁-C₄-alkylamino, di(C₁-C₄-alkyl)amino, —COR³, —COOR³, —COOH, —CONR⁴R⁵, —OCOR³, —OCOOR³, —OCONR⁴R⁵, —NR⁴COR⁵, —NR⁴COOR⁵, —NR⁴CONR⁴R⁵ and —C(═X)NHR²; or
aryl or heteroaryl, where the latter two radicals may optionally be substituted by 1, 2 or 3 radicals selected independently from among C₁-C₄-alkyl, halogen, hydroxy, C₁-C₄-alkoxy, amino, C₁-C₄-alkylamino, di(C₁-C₄-alkyl)amino, —COR³, —COOR³, —COOH, —CONR⁴R⁵, —OCOR³, —OCOOR³, —OCONR⁴R⁵, —NR⁴COR⁵, —NR⁴COOR⁵, —NR⁴CONR⁴R⁵ and —C(═X)NHR²;
R² is hydrogen; or
C₁-C₂₀-alkyl, C₂-C₂₀-alkenyl or C₃-C₇-cycloalkyl, where the latter three radicals may optionally be substituted by 1 or 2 radicals selected independently from among phenyl, halogen and C₁-C₄-alkoxy; or
aryl or heteroaryl, where the latter two radicals may optionally be substituted by 1, 2 or 3 radicals selected independently from among C₁-C₄-alkyl, halogen, C₁-C₄-alkyl, halogen and C₁-C₄-alkoxy;
R³ is C₁-C₄-alkyl;
R⁴, R⁵ are each hydrogen or C₁-C₄-alkyl;
X is O;
is reacted.

8. The process according to claim 1, wherein a compound of the formula II in which
R¹ is hydrogen or —C(═X)NHR²; or
C₁-C₂₀-alkyl, C₂-C₂₀-alkenyl or C₃-C₇-cycloalkyl, where the latter three radicals may optionally be substituted by 1, 2 or 3 radicals selected independently from among phenyl, halogen, hydroxy, C₁-C₄-alkoxy, amino, C₁-C₄-alkylamino, di(C₁-C₄-alkyl)amino, —COR³, —COOR³, —COOH, —CONR⁴R⁵, —OCOR³, —OCOOR³, —OCONR⁴R⁵, —NR⁴COR⁵, —NR⁴COOR⁵, —NR⁴CONR⁴R⁵ and —C(═X)NHR²; or
aryl or heteroaryl, where the latter two radicals may optionally be substituted by 1, 2 or 3 radicals selected independently from among C₁-C₄-alkyl, halogen, hydroxy, C₁-C₄-alkoxy, amino, C₁-C₄-alkylamino, di(C₁-C₄-alkyl)amino, —COR³, —COOR³, —COOH, —CONR⁴R⁵, —OCOR³, —OCOOR³, —OCONR⁴R⁵, —NR⁴COR⁵, —NR⁴COOR⁵, —NR⁴CONR⁴R⁵ and —C(═X)NHR²;
R² is C(═X)R⁶;
R³ is C₁-C₄-alkyl;
R⁴, R⁵ is hydrogen or C₁-C₄-alkyl;
R⁶ is hydrogen; or
C₁-C₂₀-alkyl, C₂-C₂₀-alkenyl or C₃-C₇-cycloalkyl, where the latter three radicals may optionally be substituted by 1, 2 or 3 radicals selected independently from among phenyl, halogen, hydroxy, C₁-C₄-alkoxy, amino, C₁-C₄-alkylamino, di(C₁-C₄-alkyl)amino, —COR³, —COOR³, —COOH, —CONR⁴R⁵, —OCOR³, —OCOOR³, —OCONR⁴R⁵, —NR⁴COR⁵, —NR⁴COOR⁵, —NR⁴CONR⁴R⁵ and —C(═X)NHR²; or
aryl or heteroaryl, where the latter two radicals may optionally be substituted by 1, 2 or 3 radicals selected independently from among C₁-C₄-alkyl, halogen, hydroxy, C₁-C₄-alkoxy, amino, C₁-C₄-alkylamino, di(C₁-C₄-alkyl)amino, —COR³, —COOR³, —COOH, —CONR⁴R⁵, —OCOR³, —OCOOR³, —OCONR⁴R⁵, —NR⁴COR⁵, —NR⁴COOR⁵, —NR⁴CONR⁴R⁵ and —C(═X)NHR²;
X is O;
is reacted.

9. The process according to claim 1, wherein a compound of the formula II in which
R¹ and R² together form a —(CH₂)ₙ—, —(CH₂)ₘ—Y—(CH₂)ₒ— or —(CH₂)ₚ—(CH═CH)_{q}— chain,
where
n is 2, 3, 4, 5 or 6;
m is 1, 2 or 3;
o is 1, 2 or 3;
p is 1, 2 or 3;
q is 1 or 2;
Y is O, S or N(C₁-C₄-alkyl);
or
together form a radical (a₁₂), (b₁₂) or (c₁₂) 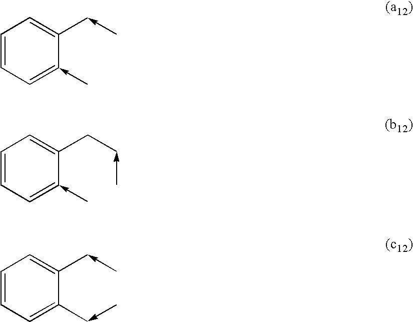
which may optionally be substituted by 1, 2 or 3 radicals selected independently from among C₁-C₄-alkyl, halogen and C₁-C₄-alkoxy;
X is O;
is reacted.

10. The process according to claim 9, wherein 2-pyrrolidone is reacted as compound of the formula II.

11. The process according to claim 1, wherein a compound of the formula II in which
R¹ and R⁶ together form a —(CH₂)ₙ—, —(CH₂)ₘ—Y—(CH₂)ₒ— or —(CH₂)ₚ—(CH═CH)_{q}— chain,
where
n is 2, 3, 4, 5 or 6;
m is 1, 2 or 3;
o is 1, 2 or 3;
p is 0, 1, 2 or 3;
q is 1 or 2;
Y is O, S or N(C₁-C₄-alkyl);
or
together form arene-1,2-diyl, naphthalene-1,8-diyl or hetarene-1,2-diyl, which may optionally be substituted by 1, 2 or 3 radicals selected independently from among C₁-C₄-alkyl, halogen and C₁-C₄-alkoxy;
or
together form a radical (a₁₆), (b₁₆) or (c₁₆) 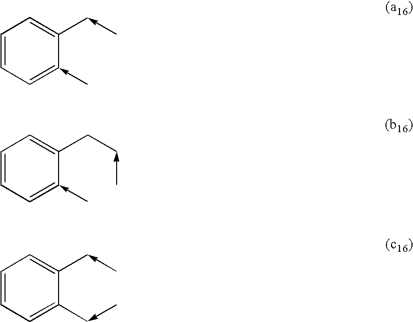
which may optionally be substituted by 1, 2 or 3 radicals selected independently from among C₁-C₄-alkyl, halogen and C₁-C₄-alkoxy;
X is O;
is reacted.

12. The process according to claim 11, wherein phthalimide is reacted as compound of the formula II.

13. The process according to claim 1, wherein the reaction is carried out at a temperature of up to 250° C.

14. The process according to claim 13, wherein the reaction is carried out at a temperature in the range from 140 to 230° C.

15. The process according to claim 1, wherein the catalyst is used in an amount of from 0.000001 to 0.0025 mol %, based on equivalents of the compound of the formula II.

16. The process according to claim 1, wherein the reaction and/or the work-up of the reaction mixture obtained by means of the reaction is/are carried out in the presence of at least one polymerization inhibitor.

17. The process according to claim 1, wherein the compound of the formula III is used in an excess of from 0.1 to 20 mol %, based on equivalents of the compound of the formula II.
